(19) United States
(12) Patent Application Publication
(10) Pub. No.: US 2006/0161073 A1
(43) Pub. Date: Jul. 20, 2006

(54) In vitro and in vivo assessment of organs and tissue and use, transplant, freshness and tissue conditions

(76) Inventors: Michael G. Singer, (US); John D. Kutzko, (US)

Correspondence Address: IRVING M. WEINER;WEINER & BURT, P.C., 635 N. US-23, P.O. BOX 186, HARRISVILLE, MI, 48740, US

(21) Appl. No.: 11/386,016

(22) Filed: Mar. 18, 2006

Related U.S. Application Data
(63) Continuation-in-part of application No. 10/701,004, filed on Nov. 4, 2003, now Pat. No. 7,003,346.
(63) Continuation-in-part of application No. 09/848,242, filed on May 3, 2001, now Pat. No. 6,587,715.
(60) Provisional application No. 60/594,200, filed on Mar. 18, 2005.
(60) Provisional application No. 60/424,828, filed on Nov. 8, 2002.

(51) Int. Cl. A61B 5/05; A61B 19/00

(52) U.S. Cl. 600/547; 128/898

(57) ABSTRACT

A method of organ and tissue vitality assessment in a biological entity, human, animal, fruit or vegetable, including the steps of: utilizing bioelectric impedance analysis in a biological model for composition analysis; and using the results of the utilizing step to provide an objective assessment of volume and distribution of fluid and tissues, and electrical health of cells and membranes of the organ or tissue.

The present patent application is a continuation-in part of and claims priority from U.S. Provisional Patent Application 60/594,200 filed Mar. 18, 2005, which in turn is a continuation-in-part of and claims priority from U.S. patent application Ser. No. 10/701,004 filed Nov. 4, 2003, now U.S. Pat. No. 7,003,346, which in turn is based on and claims priority from U.S. Provisional Patent Application Ser. No. 60/424,828 filed Nov. 8, 2002, which is a continuation-in-part of U.S. patent application Ser. No. 09/848,242 filed May 3, 2001, now U.S. Pat. No. 6,587,715. The complete disclosure of the aforementioned patent applications and patents are incorporated herein by reference thereto.

The present invention relates generally to a method and apparatus for use in the in vitro and in vivo assessment of organ and tissue vitality.

More particularly, the present invention relates to the method and apparatus mentioned above which incorporates the utilization of impedance plethysmography (IPG) bioelectrical impedance analysis (BIA) in a biological model for body composition analysis (BCA) to provide an objective assessment of an organ, tissue and/or biological entity's volume and distribution of fluids and tissue as well as the electrical health of cells and membranes; (cellular architecture).

Another aspect of the present invention relates to a method for determining illness of a biological entity, progression to death of said biological entity, and/or timing of death of said biological entity, and also relates to a method of in vivo and in vitro organ or tissue vitality assessment.

The terms “biological entity”, “patient” and “subject” as used herein mean: “any and all human beings, animals and/or living organisms, including fruits and vegetables.”

The term “non-acute death” as used herein means: “any death that does not occur acutely; it occurs more than four days (96 hours) from a precipitous event or illness; it is the end-point of a process whose duration exceeds the four-day reference; unlike that death resulting from a proximate, immediate or acute event, a ‘non-acute death’ occurs over time.”

### BACKGROUND OF THE INVENTION

The prior, but not necessarily relevant, art is exemplified by:

Bagno U.S. Pat. No. 2,111,135; Hanson U.S. Pat. No. 2,852,739; Tolles U.S. Pat. No. 3,085,566; Thomasset U.S. Pat. No. 3,316,896; Max et al. U.S. Pat. No. 3,498,288; Sigworth U.S. Pat. No. 3,882,851; Ghislaine et al. U.S. Pat. No. 4,823,804; Gallup et al. U.S. Pat. No. 5,372,141; Kotler U.S. Pat. No. 5,615,689; Brasile U.S. Pat. No. 6,024,698; Cherepenin et al. U.S. Pat. No. 6,236,866; and Kobayashi U.S. patent application Publication 2001/0023362.

The desiderata of the present invention are to avoid the animadversions of conventional methods and techniques, and to provide a novel method and apparatus for use in in vitro and in vivo assessment of organ vitality.

### SUMMARY OF THE INVENTION

A method of organ and tissue vitality in-vivo assessment comprising the steps of: placing signal introduction electrodes at/on/under the approximated skin surface location of the opposite lateral peripheral borders of said organ or tissue segment to effect the introduction of an electrical field in the organ; placing signal detection electrodes at/on/under the approximated skin surface location of the superior and inferior borders of said organ or tissue area of interest for a first part of an initial measurement of said organ or tissue region; measuring and recording first measured values of resistance and reactance and the calculation of phase angle of said organ or tissue in said initial measurement; then reversing the patient cables and clipping the said signal introduction electrodes on said electrodes superior and said inferior borders of said organ or tissue; clipping said signal detection electrodes on said electrodes opposite lateral borders of said organ; measuring and recording second measured values of said resistance and said reactance and the calculation of phase angle of said organ; and comparing said first and second values to normal values to assess vitality of said organ or tissue.

The present invention further provides a method of organ and tissue vitality assessment for transplantation of said organ or tissue being assessed, comprising the steps of: placing signal introduction electrodes at/on/under opposite lateral peripheral borders of said organ or tissue area by region in-vitro (upon harvesting) of said organ, tissue, meat, fish, fruit or vegetable; placing signal detection electrodes at/on/under superior and inferior borders of said organ or tissue or meat or fish or fruit or vegetable for a first part of an initial measurement upon said harvesting of said organ, tissue, meat, fish, fruit or vegetable; measuring and recording first measured values of resistance and reactance and calculation of phase angle of said organ or tissue, meat, fish or fruit or vegetable in said initial measurement; then reversing the patient cable of said signal introduction electrodes on said superior and said inferior borders of said organ or tissue, meat, fish or fruit or vegetable; placing said signal detection patient cable clips on said electrode previously placed at/on/under opposite lateral borders of said organ or tissue, meat, fish or fruit or vegetable; measuring and recording second measured values of said resistance and said reactance and calculation of the phase angle of said organ or tissue or meat or fish or vegetable; and comparing said first and second values to normal values to determine if said organ, tissue, meat, fish, fruit or vegetable is acceptable or not for said transplantation.

It is a primary objective of the present invention to empower the decision-maker such as a health care provider and patient with additional characterization of organ, tissue, meat, fish, fruit or vegetable vitality assessment to determine its suitability for transplantation, treatment or consumption and the response of the organ, tissue, meat, fish, fruit or vegetable in the recipient after said transplantation, treatment, storage or transport; and for detecting and characterizing the nature of illness and injury to include episodic, serious, and non-episodic chronic illness and injury, its progression and the effectiveness of treatment interventions and the prognosis of a patient and/or the freshness, viability, marketability and edibility of a biological entity; to include function, inflammation, infection and rejection of said organ and/or tissue.

In conjunction with the foregoing, the present invention also provides a method for determining the presence and degree of illness of a biological entity, progression to response to treatment, recovery or the death of said biological entity, and/or timing of death of said biological entity, comprising the steps of: taking whole body measurements of resistance, reactance, phase angle, extracellular water volume, and intracellular water volume at predetermined intervals of time; recording said whole body measurements; comparing initial values of said whole body measurements to normal values of said whole body measurements and to serially measured values of said whole body measurements; and determining, from said comparison step, hallmarks of said illness of said biological entity, said progression to said death of said biological entity, and/or said death of said biological entity.

### BRIEF DESCRIPTION OF THE DRAWINGS

FIG. 1 is a schematic illustration of one embodiment of the present invention.

FIG. 2 illustrates how electrodes may be placed on a hand for the BIA testing procedure.

FIG. 3 illustrates how the electrodes may be placed on the foot for the BIA testing.

FIG. 4 illustrates the testing methods for various portions of the body, to indicate where impedance plethysmography diagnostics fits in the testing regimen.

### DETAILED DESCRIPTION OF THE INVENTION

BIA is an electrodiagnostic methodology based upon the conductive properties of the body's tissues, cells, and fluids. The BIA instrument, such as that disclosed in U.S. Pat. No. 5,372,141, an impedance plethysmograph, may use a constant current source producing a low-voltage electrical signal, usually 800 micro-amps at a high frequency, often fixed at 50 KHz, although a range of frequencies, electrode arrays and sampling rates may be used to set up an electrical field in the whole body or a body segment using a pair of surface ECG-type or otherwise configured electrodes.

The methods of the present invention can utilize a modification of the body composition analyzer disclosed in U.S. Pat. No. 5,372,141, the entire contents of which are incorporated herein by reference thereto.

In accordance with the present invention, utilization of BIA in a biological model for BCA provides an objective assessment of the study subject's (whole body or organ (regional)) volume and distribution of fluids and tissues, as well as the electrical health of the cells and membranes.

The characteristics of BIA include precision, accuracy, feasibility and economy. BIA may be applied to any area of interest, locally, regionally or to the whole body. It is non-offensive, causing no harm. It may be repeated freely, as desired, to illustrate change over time so that progression of conditions, the response to disease and treatment intervention can be monitored and intervention modified or changed to improve the individual patient's response and outcome.

One aspect of the present invention applies the IPG/BIA technology for assessment of vitality of organs for transplant, vitality of organs from other species for human transplantation (xenotransplantation), and to monitor and assess the timing of death.

Organ vitality assessment is based upon the ability of a modified BIA for BCA to illustrate the health of cells and their membranes by the measured resistance (R), reactance (X) and calculated phase angle (φ).

Upon organ harvest, signal introduction electrodes are placed at/on/under the opposite lateral peripheral borders of the organ being assessed, and signal detection electrodes are placed at/on/under the superior and inferior borders of the organ being assessed for the first part of the initial measurement.

The values of electrical resistance (R) and impedance (X) are measured and phase angle calculated and recorded.

The signal introduction patient cable clips are then re-positioned or placed on the electrode superior and inferior borders of the organ being assessed, while the signal detection patient cable clips are now re-positioned or placed the electrode opposite lateral peripheral borders of the organ being assessed.

Further values of R and X are measured and phase angle calculated and recorded.

The values are then compared to normal values, and the organ is determined to be acceptable (vital) or not.

If acceptable (vital), prior to organ implant (transplantation or xenotransplantation), the sequence of steps described hereinabove is repeated with comparison being made to the electrical values which were measured and recorded upon organ harvest and after transplant engraftment to continue the evaluation of vitality and patient response.

The values should be within an acceptable range of agreement denoting no further loss of organ vitality, and then the implantation is completed.

In accordance with the present invention, the same scenario is utilized for organs from different species.

For determination of the timing of death, whole body measurements are made at predetermined intervals of time (preferably, but not necessarily, every other day) with electrical resistance (R), reactance (X), and phase angle (φ) being measured and recorded. Frequency of measurement varies in proportion to the events being captured to include the progression of the underlying disease processes, the treatment intervention/s madeand the normal changes of physiology. Initial values are compared to normal values and to those serially measured and recorded.

The uncorrectable loss of cell mass and membrane capacity, as evidenced by a reduction in X and φ or by an uncorrectable and increasing disparity of ECW (extracellular water) volume being greater than ICW (intracellular water) volume and remaining uncorrectable, are the hallmarks of the progression to the death of the biological entity.

φ values consistently less than ˜four degrees denote serious illness.

φ values consistently less than ˜two degrees denote imminent demise.

One embodiment of the present invention provides a method for determining illness of a biological entity, progression to death of said biological entity, and/or timing of death of said biological entity, comprising the steps of: taking whole body measurements of resistance, reactance, phase angle, extracellular water volume, and intracellular water volume at predetermined intervals of time; recording said whole body measurements; comparing initial values of said whole body measurements to normal values of said whole body measurements and to serially measured values of said whole body measurements; and determining from said comparison step hallmarks of said illness of said biological entity, said progression to said death of said biological entity, and/or said death of said biological entity.

Another embodiment of the present invention provides a method of organ vitality assessment for transplantation of said organ being assessed, comprising the steps of: placing signal introduction electrodes at/on/under opposite lateral peripheral borders of said organ upon harvesting of said organ; placing signal detection electrodes at/on/under superior and inferior borders of said organ for a first part of an initial measurement upon said harvesting of said organ; measuring and recording first measured values of resistance and reactance and calculation of phase angle of said organ in said initial measurement; then placing said signal introduction patient cable clips on the electrode at said superior and said inferior borders of said organ; placing said signal detection patient cable clips on said electrode at/on/under opposite lateral borders of said organ; measuring and recording second measured values of said resistance and said reactance and calculation of phase angle of said organ; and comparing said first and second values to normal values to determine if said organ is acceptable or not for said transplantation.

There will now be described one embodiment of the present invention. This embodiment provides a method and apparatus for use in detecting the presence and severity of illness, the effectiveness of treatment interventions, and the ability to change treatment to be more effective or aggressive; to optimize outcome, limit morbidity and mortality and illustrate the patient's prognosis.

The purpose of this embodiment is to empower the healthcare provider and the patient by detecting and characterizing the presence and nature of illness and injury to include episodic, serious, and non-episodic chronic illness and injury, its progression, and the effectiveness of treatment interventions and the prognosis of the patient.

There is provided a method and system for use in detecting the presence and severity of illness in diagnosing and treating a patient to optimize the treatment intervention and determine the prognosis of the patient.

This system employs the use of Whole Body Impedance Analysis to measure the patient's Resistance, Reactance, Phase Angle, and related electrical values at a healthy baseline, and thereafter in relation to the patient's complaints to evaluate the temporal or progressive nature of negative values or diminution of the measured values over time.

Specifically, the system identifies the patient's healthy baseline measured electrical values and, during routine health examinations or when the patient complains of any symptoms or experiences any signs of illness or injury, illustrates excursion from the baseline values that may exceed a thirty-day time frame or progressively diminish. Episodic illness and recoverable injury is characterized by a brief, less than thirty days, excursion below the baseline values and return to the baseline values. More severe illness, chronic disease and injury are characterized by progressive or rapid diminution of the measured values.

Once an effective treatment intervention is begun, the measured values will stabilize and then return to the baseline values indicative of the patient's positive prognosis. More effective treatment is indicated by a more rapid return to baseline-measured values. If the values do not improve, a modified or more aggressive treatment intervention is indicated whose positive effectiveness will be indicated by the initial stabilization of the measured values and their subsequent return to baseline values. Prognosis is proportional to the speed and direction of the return of the measured value to or from the baseline values. A positive prognosis is indicated by a progressive and/or rapid return to the measure baseline values. A negative prognosis is indicated by a progressive and/or rapid diminution of the measured values. The speed of loss or gain of the measured values is proportional to the return of health or the severity of the illness or injury. A neutral or stabilized measured value lower than the healthy baseline, over an extended period of time, greater than six months, indicates a new baseline, a less healthy condition and predisposition to future illness.

Frequency of measurements is in proportion to the severity of the process to be illustrated; more severe illness or injury, characterized by more severe symptoms, signs and negative laboratory findings and progressive and/or rapid diminution of the measured values, require more frequent measurements, daily and every other day. Less severe illnesses and injuries may be illustrated with weekly measurements.

The invention will now be further explained with reference to FIGS. 1-3.

The primary study method for an impedance plethsymographic examination either Whole-Body 1 or Regional 2 is simple and straightforward. The patient requires no advanced preparation for the study. However, the patient should not be diaphoretic, soaked in urine or any other surface liquid that would provide an alternative pathway for the conduction of the electrical signal that is the basis of the study.

The patient is counseled to lie quietly, motionless, and informed that the test will take less than five minutes if the patent is cooperative. The patient is generally placed in a supine position with arms and legs abducted about thirty degrees from the midline on a dry non-conductive surface. Whole Body 1 and Regional 2 studies require a tetrapolar electrode scheme in which placement of four (two pairs) surface, ECG electrodes in strict relation to anatomical landmarks at the wrist and ankle. If the patient's skin is either too dry or too oily, wiping the electrode placement area with an alcohol prep wipe is suggested. The right side of the body is generally used with the electrodes placed ipsilaterally. However if the patient's condition requires contra-lateral placement and alternative body positions, they can be utilized with the understanding and proviso that the same position will be repeated with all future measurements. The signal detection (SD) electrodes 3 or 4 must be placed with the greatest precision in relation to known anatomical landmarks on both the wrist and the ankle.

On the wrist, the superior linear border of the electrode, its top straight line, must equally bisect the ulnar stylus, bone prominence (bump) on the little finger side of the wrist with the tab of the electrode facing away from the body of the patient. The signal introduction (SI) electrodes 5 are placed distal from the SD electrodes 3 and must be kept at a minimum distance that equals or exceeds that of the diameter of the segment being measured (e.g., the wrist). This is most easily and efficiently accomplished by using the distal phalanx of the middle finger, just proximal to the nail.

On the ankle, the SD electrode 4 is placed so that the superior linear border equally bisects the medial malleolous (the bump on the big toe side of the ankle) with the tab facing outwards from the patient. Care should be exercised to use the medical malleolous because the lateral malleolous (the bump on the little toe side of the ankle) is inferior or below the medial malleolous landmark. The SI electrode 6 is placed on the big toe, as shown in FIG. 1.

The plethysmograph is connected via patient cable leads with strict attention paid to SI and SD leads connected to SI and SD electrodes. The device is energized and the values of resistance and reactance in ohms, are measured individually, allowing a moment (ten to fifteen seconds) to settle, and then are recorded. The electrodes are carefully removed so as not to injure friable skin or contaminate the examiner.

Any standard impedance plethysmograph that utilizes a 500-800 micro-amp constant current electrical source at 50-kilohertz frequency can be utilized. Preferably, but not necessarily, an RJL Systems, Inc. manufactured instrument system may be used for both Whole Body 1 and Regional 2 measurements.

For Regional 2 measurements, the patient is prepared in the same manner as with a Whole-Body 1 examination. For in-vivo Regional 2 measurements of the chest, abdomen or extremities (arms/legs, left-right, upper or lower), the signal detection electrodes 7 are placed superiorly and inferiorly in precise relation to the area of interest. The distance between the detection electrodes is precisely measured and recorded in centimeters. The skin is marked with a surgical pen to assure accurate and reproducible electrode placement for serial measurements. The SI electrodes 1 are best placed in the standard Whole-Body locations, however this requires a specialized patient cable with adequate distance or throw, about eighteen inches of length allowed, between the insertion point into the patient cable to and from the clip ends. The impedance plethysmograph is connected via the patient cables with strict adherence to the SD lead to the SD electrode and the SI lead to the SI electrode. The measured values are recorded and the electrodes carefully removed.

The measured values, resistance, reactance and phase angle (calculated) are recorded, archived and graphically presented, compared to normal values and then followed serially to illustrate change over time and illuminate the processes of disease progression and response to treatment. The frequency of serial measurements is proportional to the dynamic of the event to be captured. If at all possible, a baseline study value is particularly desirable.

Disorders characterized by dynamic shifts of extracellular fluid volumes require more frequent measurements, often prior to and after a procedure or treatment such as a patient requiring hemodialysis, aggressive diuresis in organ failure or repletion of fluids in acute dehydration or trauma. The measured resistance value in ohms is inversely proportional to the extracellular fluid volume of the patient. When resistance ohms decrease fluid has increased and conversely when resistance ohms increase fluid volume has decreased. So, once an initial ohm measurement value is established by baseline or first study, subsequent measurements illustrate the patient's course and response to disease progression and the effectiveness of the selected treatment intervention. The severity of the disease or insult condition evidenced by the speed of the excursion from baseline or initial measurement value. Fluid changes that move more than fifty ohms in a twenty-four hour period are severe and indicate a more acute and serious condition than those that move fifty ohms in a week's time indicative of a more chronic condition. Both conditions require intervention, however as chronic insidious changes are as adverse to survival as more rapid changes. These changes may be evidenced in both Whole Body 1 and Regional 2 measurements. Whole Body 1 measurements are more general in their value, indicative of conditions and events that encompass the organism as a whole such as cardiac or renal failure and acute dehydration. Regional 2 measurements provide a site-specific assessment of fluid volumes such as those found with pleural effusion in the chest, ascites in the abdomen or even cerebral edema. The changes of measured electrical values precede changes seen on x-ray, physical examination, or from laboratory studies.

Once again, increasing ohms of resistance indicate a drying and fluid reduction while decreasing ohms of resistance indicate increased fluid volumes. Thoracic resistance values that are increasing indicate a drying chest and conversely decreasing resistance values indicate additional accumulation of fluid. These changes clearly indicate the improvement or worsening of disease conditions and the individual's response to treatment and ergo, its effectiveness. The extent and aggressiveness of therapy can be altered and modified to “optimize” the beneficial effects.

Reactance values are proportional to the number and integrity (health) of cell wall membranes so when cells increase or decrease reactance values follow. The cells that change in this manner are those of the somatic and visceral protein tissues, such as skeletal musculature organs such as the liver, spleen, lungs, heart stomach and intestines. Cellular alterations are generally slower to occur and are affected by metabolic and specific disease processes (inflammation, infection, rejection and/or chemical imbalances, trauma, insult and/or injury. However, overly aggressive diuresis, excessive hemodialysis or cellular targeted pathologies such as Rhabdomyolysis can all result in rapid, days versus a week, changes in cell mass, membrane status and measured reactance values. Excursions from the baseline or initial measurement value indicate the type and progression of disease and/or the effectiveness of treatment interventions. Increased cells (membranes) and anabolic metabolism are evidenced by a rise in the ohms of reactance, generally a sign of improvement. A slowly decreasing ohm value of reactance indicates a negative or catabolic metabolism condition. A more precipitous and rapid decrease in reactance is indicative of unique conditions that rapidly affect cells and their membranes, such as the effect of Rhabdomyolysis skeletal muscle or rejection or infection of an organ system.

Regional measurement values of ohms of reactance are used for these disease specific investigations while whole body values are used for the assessment of metabolic evaluation.

A derivative of the measured values of resistance and reactance is the arc tangent of reactance to resistance expressed in degrees or Phase Angle. Phase Angle is the cumulative expression of the changes and ratios of cell mass and extracellular fluid that result from disease, insult and/or treatment intervention and can by itself be used to gauge the severity and progression of pathologies and the effectiveness and benefits of treatment. As such the phase angle reflects the condition of the cell membrane and its mediation between the intra and extracellular milieus. A positive prognosis or more healthy and vital organ is indicated by an increasing phase angle while a poor prognosis or less vital or healthy organ is associated with a phase angle decrease. Phase angle has been correlated with survival and the timing of non-acute death. Phase angle can be derived from both whole body and regional measurements and followed serially to establish prognosis.

Treatment interventions can be measured for their effectiveness on the individual patient by following phase angle. More effective treatments are evidenced by an increasing phase angle while those less effective are seen as producing little or no increase. Once phase angle persistently degrades to and stays below four degrees, the patient is seriously ill and treatment should be aggressive and modified to be effective and optimal. If phase angle does not stabilize or increase through multiple iterations of treatment, a curative or restorative treatment goal outcome is doubtful. A phase angle of persistently less than two degrees is associated with pending and unavoidable mortality and a need for discontinuation of curative or restorative treatment effort and for the initiation of palliative treatment, care and comfort. Admission to a hospice can be objectively based upon phase angle monitoring providing the patient with improved end-of-life care and comfort.

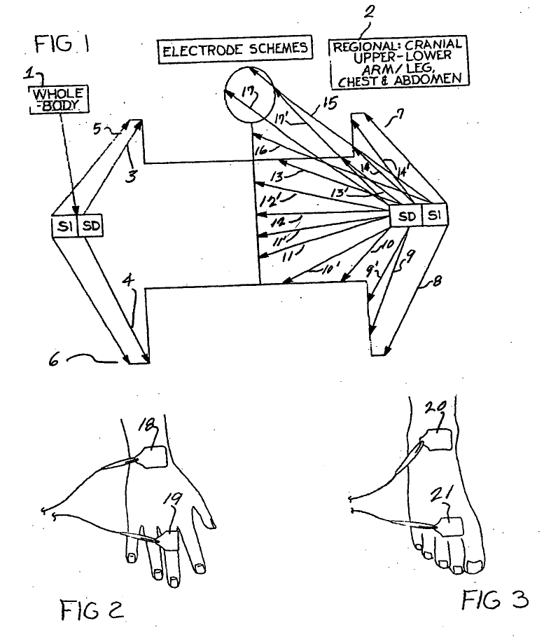

FIG. 2 illustrates how electrodes may be placed on the hand for the BIA Testing Procedure.

The detecting electrode edge 8 is placed on an imaginary line bisecting the ulna head (bone on little finger side of wrist)

The signal electrode 9 is placed on the first joint of the middle finger.

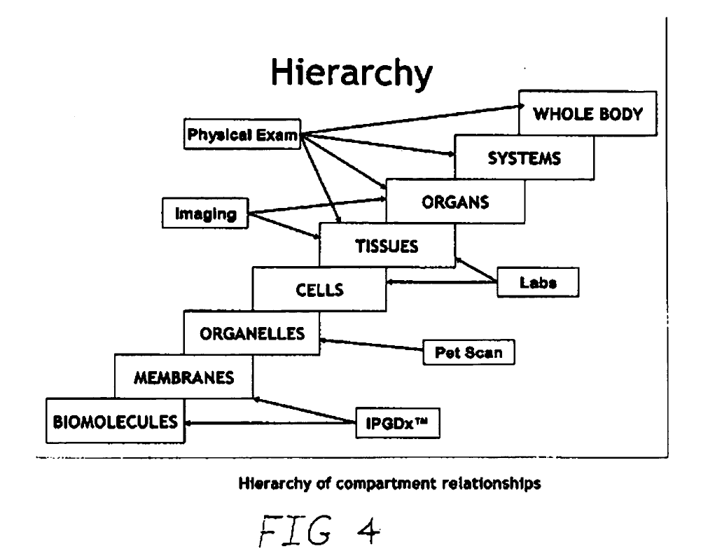

FIG. 3 illustrates how electrodes may be placed on the foot.

The detecting electrode edge 10 is placed on an imaginary line bisecting the medial mellealus (bone on big toe side of ankle).

The signal electrode 11 is placed on the base of the second toe.

The exam area should be comfortable and free of drafts. The exam table surface must be non-conductive and large enough for the subject to line supine with the arms 30 degrees from the body, and legs not in contact with each other.

The subject should not have exercised or taken a sauna within 3 hours of the study. The subject's height and weight should be accurately measured and recorded. The subject should lie quietly during the entire test. The subject should not be diaphoretic or wet from sweat or urine. The subject should not have a fever or be in shock or if such is present comparison to serial measurements should be made only to those made in the same or similar conditions. The study and testing procedure should be explained to the subject.

The subject should remove the shoe and sock and any jewelry on the electrode side (generally the study is completed on the right side of the body). The body side (left or right) should always be used subsequently.

The subject should lie supine with the arms 30 degrees from the body with legs not touching.

The electrode sites may be cleaned with alcohol, particularly if the skin is dry or covered with lotion.

The electrodes and patient cables are attached as shown in FIGS. 2 and 3.

The analyzer is turned on, making sure the subject refrains from moving. When the measurements have stabilized, record the displayed Resistance (R) and Reactance (Xc) with the subject's name, age, gender, height and weight.

The entire testing time is less than 5 minutes—the BIA analyzer is on for less than one minute.

The results are available immediately from the software program.

The study may be repeated as often as necessary.

The present invention also embraces the features of using the invention for various areas of interest, for example, whole-body thoracic, abdominal, extremity, etc.

Impedance plethysmography diagnostics (IPGDX™) are based upon the illustration of “cellular” level physiology through their measured electrical equivalents. The subject becomes the only unknown part of an electrical circuit.

Based upon the purpose of the study the patient's whole-body or a regional section will be studied. A four-electrode tetrapolar scheme of two pairs of surface ECG-type stick-on electrodes is placed in relation to prominent and/or carefully noted anatomical landmarks.

One pair introduces the electrical field; the “signal introduction” electrodes. The second pair detect the changes in the electrical field that result from the patient being part of the circuit and are placed in relation to the area of interest either whole-body or regional.

A patient cable is connected to the electrodes when necessary the patient cables are moved from signal introduction electrodes to signal detection electrodes to make the second measurement of a regional measurement or in-vitro organ assessment and to the plethysmograph. The plethysmograph has two purposes; first to generate a constant precise electrical signal and to measure the ‘patient segment’ of the circuit.

The electrical signal may beat a fixed or variable frequency. The voltage is generally fixed at ˜500 to 800 micro-amps.

The frequency is maintained above the threshold that would stimulate, disturb or insult the tissues of the subject. The signal strength is maintained at a constant value to accommodate subjects of various physiognomies.

The measured values of electrical resistance (R) and Reactance (Xc) are measured and recorded along with patient identification, age, gender, height, weight and if a regional measurement is performed the distance between the detection electrodes and the area of interest is identified.

The distance between the detection electrodes is important as the area of interest must be between the detection electrodes and they must be configured accordingly to provide the depth of measurement appropriate to the phenomenon sought or captured. A peripheral event in the skin such as capillary perfusion is seen with the detection pair of electrodes close to each other. The study of an internal structure requires the distance between the electrodes to be increased to address its anatomical location.

For instance in studying the liver two pairs of detecting electrodes would be used to that would approximate the superior/inferior borders and the lateral/medial borders to record measured values from the entire organ. The signal introduction electrodes must be at least the distance from the detection electrodes that is greater than the diameter of the segment of the body they are applied to.

They are best kept on the hand and foot but may be applied superiorly and inferiorly to the area of interest as long as they are at a distance greater than the diameter of the body segment. This is simply due to the need for the electrical field to be fully and adequately distributed through the area of interest to complete the circuit and include the area of interest within the detection electrode array.

From the measured values of R and Xc the Phase Angle (Pa) is calculated; the arc tangent relationship of Xc to R expressed in degrees. The measured R and Xc are a series circuit model and are transformed mathematically to the equivalent parallel circuit model of the body.

The electrical values of R, Xc and Pa correspond to physiologic variables of biology. The R value is inversely proportionate to extracellular water.

The Xc value is proportional to cell mass, as the plasma bi-lipid membrane acts as a capacitor and reflects the intracellular water volume and body cell mass (combined somatic and visceral proteins). A single measurement is essentially a ‘snap-shot’ in time of the conditions encountered.

The measured values may be compared to ‘normal’ and assessment of excess, equality or absence can be made. Through serial assessments change over time can be documented.

The technique is highly reproducible as it is a simple electrical circuit, which does not change and is well understood, while the subject part of the circuit is constantly changing, so the changes in the measured values are inherent to those of the subject.

A small error is possible with misplacement of the detection electrode pair by the examiner; thusly prominent anatomical landmarks, measured values and simply marking the skin can be used to minimize this effect. This operator error is ˜2% or less and is managed through training, testing and specialized electrode arrays.

The technique is best suited to illustrate change over time as the condition of interest may change; such as disease progression or the response to treatment interventions. In this manner the results become guides to assessing the effectiveness of treatment, the effects that changes in the treatment intervention may induce and the patients overall response. The particular value of the results is that they are cellular level values.

With reference to FIG. 4, consider that the body is organized in an ensemble of compartments and that this hierarchy of organized functionally and spatially distinct compartments range from the microscopic (intracellular) to macroscopic levels (gross whole body). The transport process and communication between each level is mediated through cell membranes.

On a microscopic level physiologic interactions are mediated through channels, carriers and pumps; on the macroscopic level by skeletal musculature (somatic body cell mass). Pathophysiology from any etiology; insult, injury or disease process is evidenced on the membrane transport system gone awry.

The data resulting from the impedance measurement is more sensitive, specific and valuable than traditional indices because it is the precursor to these ‘down-stream’ occurrences. This membrane level dataset provides an invaluable bridge seemingly prescient as changes at this level of the hierarchy occur to those downstream.

Prior to a change in a blood chemistry value, the development of inflammation, infection, rejection or the prominence of a physical sign, finding on an imaging study or patient complaint of a symptom a membrane transport process is askew. This change can be noted through the impedance study and correlated with the more gross and later developing findings and be used to provide better interventions sooner.

IPGDX™ test results provide information about;
Fluid volumes and shifts between the intra and extracellular milieu
Nutrition status
Cell membrane health
Metabolism
Infection
Inflammation

The cellular architecture of
Organs
Muscles

These data are able to be used to evaluate;
Presence of disease
Locally
   Systemic
   Regionally
Progression of disease
Response to pharmacologic treatment intervention
Need to change or terminate treatment
Patient's prognosis
Organ vitality and function
In vivo
Hepato-cellular architecture
Cirrhosis
Fibrosis
Steatosis
Lung water (Pulmonary edema)
In vitro
Organs for transplant

Cellular architecture

Timing of non-acute death

Outcome

Classification of potential treatment outcome

Curative

Restorative

Palliative

The foregoing lists are not inclusive, but are intended simply to show examples of the use of the present invention.

The present invention covers not only in vitro transplantation applications, but it also covers impedance In vivo assessment of organ vitality, e.g., liver (kidney).

With the patient in a dorsal recumbent position; lying on their back on a non-conductive surface; Standard whole-body measurement is made with signal introduction electrodes placed on the distal Right Hand and Foot, detection electrodes placed in relation to ulnar stylus at wrist and medial malleolous in ankle; measurement of R and Xc taken and recorded

Detection electrodes are placed in relation to superior/inferior borders of liver and lateral/medial borders of liver measurement of R and Xc taken and recorded from each set.

The measured values are converted to their equivalent parallel circuit model and phase angle is calculated, they are compared to “normal” values and previously measured values if available over time as they change in response to treatment and disease progression.

The presence of pathophysiology such as; cirrhosis, fibrosis and/or steatosis or ascites is evidenced by the measured values. As opposed to liver biopsy the impedance assessment is noninvasive, samples the entire organ (versus 1/50,000 h ) and is without complication (versus a rate of 0.59%).

The present invention also provides that once organ, tissue, meat, fish, fruit or vegetable is transplanted or treated into recipient, follow-up measurements of whole-body and regional resistance, reactance and the calculation of phase angle (specific to the site of transplant of said organ tissue, meat, fish, fruit or vegetable) will be made with electrodes placed for a whole-body measurement (wrist and ankle, top and bottom, left and right) and with the detection electrodes superior and inferior as well as medial lateral to the organ or tissue site.

Although the invention has been described in detail in the foregoing only for the purpose of illustration, it is to be understood that such detail is solely for that purpose and that variations and modifications can be made therein by those of ordinary skill in the art without departing from the spirit and scope of the invention including all equivalents thereof.

1. A method of organ and tissue vitality assessment in a biological entity, human, animal, fruit or vegetable, comprising the steps of:
utilizing bioelectric impedance analysis in a biological model for composition analysis; and
using the results of said utilizing step to provide an objective assessment of volume and distribution of fluid and tissues, as well as electrical health of cells and membranes of said organ or tissue of any biological entity.

2. The method according to claim 1, including the step of:
utilizing a modified bioelectric impedance analysis for composition analysis to assess the health of cells of said organs and tissues or the biological entity by the measured reactance thereof.

3. A method according to claim 1, wherein:
upon harvesting, treating or transporting said organ or tissue or meat, fish, fruit or vegetable from the donor or source, including the steps of:
placing signal introduction electrodes on opposite lateral peripheral borders of said organ tissue, meat, fish, fruit or vegetable;
placing signal detection electrodes at superior and inferior borders of said organ tissue, meat, fish, fruit or vegetable for a first part of an initial measurement;
measuring and recording first values of resistance and reactance and calculating the phase angle of said organ tissue, meat, fish, fruit or vegetable in said initial measurement;
then placing said signal introduction electrodes on said superior and said inferior borders of said organ tissue, meat, fish, fruit or vegetable;
placing said signal detection electrodes on said opposite lateral borders of said organ tissue, meat, fish, fruit or vegetable;
measuring and recording second values of said resistance and said reactance and calculating the phase angle of said organ tissue, meat, fish, fruit or vegetable; and
comparing said first and second values to normal values to assess vitality.

4. A method according to claim 2, wherein:
upon arrival of said organ tissue, meat, fish, fruit or vegetable at the location of the recipient including the steps of:
placing signal introduction electrodes on opposite lateral peripheral borders of said organ, tissue, meat, fish, fruit or vegetable;
placing signal detection electrodes at superior and inferior borders of said organ, tissue, meat, fish, fruit or vegetable for a first part of an initial measurement of said organ, tissue, meat, fish, fruit or vegetable;
measuring and recording first values of resistance and reactance and calculating the phase angle of said organ, tissue, meat, fish, fruit or vegetable in said initial measurement;
then placing said signal introduction electrodes on said superior and said inferior borders of said organ, tissue, meat, fish, fruit or vegetable;
placing said signal detection electrodes on said opposite lateral borders of said organ, tissue, meat, fish, fruit or vegetable;
measuring and recording second values of said resistance and said reactance and calculating the phase angle of said organ, tissue, meat, fish, fruit or vegetable; and
comparing said first and second values to normal values to assess vitality of said organ, tissue, meat, fish, fruit or vegetable.

5. A method according to claim 3, wherein:
prior to the implantation, treatment and/or consumption of said organ tissue, meat, fish, fruit or vegetable into the recipient including the following additional steps:
again placing said signal introduction electrodes on said opposite lateral peripheral borders of said organ tissue, meat, fish, fruit or vegetable;
again placing said signal detection electrodes at said superior and said inferior borders of said organ tissue, meat, fish, fruit or vegetable;
measuring and recording third values of resistance and reactance and calculating the phase angle of said organ tissue, meat, fish, fruit or vegetable;
then again placing said signal introduction electrodes on said superior and said inferior borders of said organ tissue, meat, fish, fruit or vegetable;
again placing said signal detection electrodes on said opposite lateral borders of said organ tissue, meat, fish, fruit or vegetable;
measuring and recording fourth values of said resistance and said reactance of said organ tissue, meat, fish, fruit or vegetable; and
comparing said first and second values to said third and fourth values to determine if the values are within a predetermined acceptable range of agreement denoting no further loss of said organ tissue, meat, fish, fruit or vegetable vitality.

6. A method according to claim 4, including the following additional steps:
again placing said signal introduction electrodes on said opposite lateral peripheral borders of said organ tissue, meat, fish, fruit or vegetable;
again placing said signal detection electrodes at said superior and said inferior borders of said organ tissue, meat, fish, fruit or vegetable;
measuring and recording third values of resistance and reactance and calculating the phase angle of said organ tissue, meat, fish, fruit or vegetable;
then again placing said signal introduction electrodes on said superior and said inferior borders of said organ tissue, meat, fish, fruit or vegetable;
again placing said signal detection electrodes on said opposite lateral borders of said organ tissue, meat, fish, fruit or vegetable;
measuring and recording fourth values of said resistance and said reactance and calculating the phase angle of said organ tissue, meat, fish, fruit or vegetable; and
comparing said first and second values to said third and fourth values to determine if the values are within a predetermined acceptable range of agreement denoting no further loss of said organ tissue, meat, fish, fruit or vegetable vitality.

7. A method of organ, tissue, meat, fish, fruit or vegetable vitality assessment, comprising the steps of:
placing signal introduction electrodes on opposite lateral peripheral borders of said organ, tissue, meat, fish, fruit or vegetable;
placing signal detection electrodes at superior and inferior borders of said organ, tissue, meat, fish, fruit or vegetable for a first part of an initial measurement of said organ tissue, meat, fish, fruit or vegetable;
measuring and recording first values of resistance and reactance of said organ, tissue, meat, fish, fruit or vegetable in said initial measurement;
then placing said signal introduction electrodes on said superior and said inferior borders of said organ tissue, meat, fish, fruit or vegetable;
placing said signal detection electrodes on said opposite lateral borders of said organ, tissue, meat, fish, fruit or vegetable;
measuring and recording second values of said resistance and said reactance of said organ, tissue, meat, fish, fruit or vegetable; and
comparing said first and second values to normal values to assess vitality of said organ, tissue, meat, fish, fruit or vegetable.

8. A method according to claim 7, including the following additional steps:
again placing said signal introduction electrodes on said opposite lateral peripheral borders of said organ, tissue, meat, fish, fruit or vegetable;
again placing said signal detection electrodes at said superior and said inferior borders of said organ, tissue, meat, fish, fruit or vegetable;
measuring and recording third values of resistance and reactance and calculating the phase angle of said organ, tissue, meat, fish, fruit or vegetable;
then again placing said signal introduction electrodes on said superior and said inferior borders of said organ, tissue, meat, fish, fruit or vegetable;
again placing said signal detection electrodes on said opposite lateral borders of said organ, tissue, meat, fish, fruit or vegetable;
measuring and recording fourth values of said resistance and said reactance and calculating the phase angle of said organ, tissue, meat, fish, fruit or vegetable; and
comparing said first and second values to said third and fourth values to determine if the values are within a predetermined acceptable range of agreement denoting no further loss of said organ, tissue, meat, fish, fruit or vegetable vitality.

9. A method of organ, tissue, meat, fish, fruit or vegetable vitality assessment for transplantation, treatment, consumption or transport of an organ, tissue, meat, fish, fruit or vegetable being assessed, comprising the steps of:
placing signal introduction electrodes on opposite lateral peripheral borders of said organ, tissue, meat, fish, fruit or vegetable upon harvesting of said organ tissue, meat, fish, fruit or vegetable;
placing signal detection electrodes at superior and inferior borders of said organ tissue, meat, fish, fruit or vegetable for a first part of an initial measurement upon said harvesting of said organ, tissue, meat, fish, fruit or vegetable;
measuring and recording first values of resistance and reactance of said organ, tissue, meat, fish, fruit or vegetable in said initial measurement;
then placing said signal introduction electrodes on said superior and said inferior borders of said organ, tissue, meat, fish, fruit or vegetable;
placing said signal detection electrodes on said opposite lateral borders of said organ, tissue, meat, fish, fruit or vegetable;
measuring and recording second values of said resistance and said reactance of said organ, tissue, meat, fish, fruit or vegetable; and
comparing said first and second values to normal values to determine if said organ, tissue, meat, fish, fruit or vegetable is acceptable or not for said transplantation, consumption, treatment or transportation effects.

10. A method according to claim 9, wherein:
if said organ, tissue, meat, fish, fruit or vegetable is acceptable, then prior to implanting, consuming or further treatment said organ, tissue, meat, fish, fruit or vegetable, performing the following steps;
again placing said signal introduction electrodes on said opposite lateral peripheral borders of said organ, tissue, meat, fish, fruit or vegetable;
again placing said signal detection electrodes at said superior and said inferior borders of said organ, tissue, meat, fish, fruit or vegetable for a first part of an initial post-harvest (transport/treatment)/pre-implant consumption, treatment or transport measurement;
measuring and recording third values of resistance and reactance of said organ, tissue, meat, fish, fruit or vegetable in said initial post-harvest (transport, treatment)/pre-implant measurement;
then placing said signal introduction electrodes on said superior and said inferior borders of said organ, tissue, meat, fish, fruit or vegetable;
placing said signal detection electrodes on said opposite lateral borders of said organ, tissue, meat, fish, fruit or vegetable;
measuring and recording fourth values of said resistance and said reactance of said organ, tissue, meat, fish, fruit or vegetable; and
comparing said first and second values to said third and fourth values to determine if the values are within a predetermined acceptable range of agreement denoting no further loss of said organ, tissue, meat, fish, fruit or vegetable vitality.

11. A method according to claim 9, including:
harvesting said organ from a first species of biological entity; and
implanting said organ in a different species of biological entity.

12. A method according to claim 10, including:
harvesting said organ from a first species of biological entity; and
implanting said organ in a different species of biological entity.

13. A method according to claim 3, wherein:
said measured values of resistance and reactance and the calculation of phase angle changes will be compared to their previous values and considered in the rate of change either increase or decrease the assessment of fluid volumes, cellular architecture, freshness and vitality.

14. A method according to claim 3, including the steps of:
comparing and assessing homogeneity within heterogeneous populations based upon comparative values of calculated phase angles.

15. A method according to claim 3, wherein:
the severity, criticality or burden of an adverse condition is based upon said calculated phase angle value in that a higher value indicates a less severe, critical or burden of adversity and a lower value indicates a greater severity, criticality or burden of adversity.

16. A method according to claim 15 wherein:
the resources allocated or required to manage said adverse condition are based upon said calculated phase angle value in that the lower phase angle value entity requires greater resources than that of an entity with a greater phase angle value.

17. A method according to claim 16, wherein:
in those entities that experience a transient reduction of said phase angle value that does not fully return to the previous baseline phase angle value after apparent recovery that that entity is not fully recovered and may be predisposed to further adversity and require additional care and intervention.

18. A method according to claim 10, wherein:
the vitality of said organ will have different levels of vitality based upon its measured resistance, reactance and calculated phase angle which while it may not be optimal will be sufficient for its purpose and may further be used to classify its use for a corresponding recipient with the matching of a higher phase angle value to the recipient with a lower phase angle value and conversely the matching of a organ with a lower phase angle value with a recipient of a higher phase angle value.

19. A method according to claim 3, wherein: the freshness of a consumable biological foodstuff such as a meat, fish, fowl, fruit or vegetable is based upon said calculated phase angle value in which the higher said phase angle value as related to that value upon initial harvest or processing is compared to that level after transport or upon purchase or process for purchase.

20. A method according to claim 19, wherein:
said phase angle is used as a freshness indicator of said consumable biological foodstuff.
